(19) United States
(12) Patent Application Publication
(10) Pub. No.: US 2020/0123494 A1
(43) Pub. Date: Apr. 23, 2020

(54) METHOD FOR OBTAINING PROTEIN FROM WHEY OR MOLASSES

(71) Applicant: Proteo Alimentaria, S.A.P.I. de C.V., Ciudad de México (MX)

(72) Inventor: Rafael DE LA HUERTA BENITEZ, Ciudad de México (MX)

(73) Assignee: Proteo Alimentaria, S.A.P.I. de C.V., Ciudad de México (MX)

(21) Appl. No.: 16/623,106

(22) PCT Filed: Nov. 27, 2017
(86) PCT No.: PCT/MX2017/050024

§ 371 Date: Dec. 16, 2019

(30) Foreign Application Priority Data
Jun. 16, 2017 (MX) MX/A/2017/008026

(51) Int. Cl. C12N 1/16; A23J 1/18

(52) U.S. Cl. CPC C12N 1/16; A23J 1/18

(57) ABSTRACT

A process to ferment lactose and molasses, to obtain unicellular biomass is disclosed. The described process allows obtaining a product of high protein value which will be in the range of 50 to 90% for its application in the food industry. The process uses as a means of propagation whey of cheese or molasses, for the use by Kluyveromyces marxianus to obtain unicellular biomass and subsequently unicellular protein.

### CROSS-REFERENCE TO RELATED APPLICATIONS

This application is a National Stage application of International Application No. PCT/MX2017/050024 filed on Nov. 27, 2017, which claims priority of Mexican (MX) application Serial Number MX/a/2017/008026 filed on Jun. 16, 2017, both of which are incorporated herein by reference in their entireties.

### DESCRIPTION

### Object of the Invention

It is a purpose of the invention that is disclosed in terms of the present document, to protect a process for fermenting lactose and molasses, for obtaining a single cell biomass, the disclosed process allows obtaining a high protein value product which will be in the range of 50 to 90% for application in the food industry.

### BACKGROUND AND SUMMARY

Biotechnology is an interdisciplinary and applied branch of the science that binds chemistry, biology, biochemistry, engineering and microbiology in order to overcome practical problems, involving an optimization of cost and yields. One of the primary objectives of biotechnology is to improve the handling and use of large quantities of organic waste of agricultural origin, attempting to search for alternatives to convert these pollution sources into useful derived materials from an economical and industrial standpoint. One of the most important tangents of biotechnology focused to address this problem is the use of microorganisms as part of industrial processes, where inexpensive or industrial waste substrates are used as an energy source for the selected microorganisms to synthesize new commercial value compounds. Many are the derived organic compounds obtained by biotechnological exploitation of waste substrates. Some are obtained in the form of final or intermediate products of the microbial metabolic activity which are excreted in the medium or extracted industrially after the cell (methanol, methane, ethanol, B Complex vitamins, lactic and glutamic acid, carotenoids, xylitol, etc.). It is also feasible that the product itself be the microbial biomass as such and which constitutes a high protein content food source.

In the last part of the nineteenth century, the microorganisms used for the production of foods (bakery, preparation of alcoholic beverages, dairy industry, etc.) were first isolated in pure crop.

This development led rapidly to a better knowledge of the relationships between certain microorganisms, their activities and their products (eg, amino acids, vitamins, alcohol, etc.). Currently, with the large advances in biotechnology, the microorganisms are increasingly replacing the traditional procedures such as, for example, the production of enzymes and vitamins. This area is of great interest, since, in a near future, we will find the problem of the lack of proteins for the human and animal feed, since the population expands rapidly, but the growing soil and cattle are not.

One of the possibilities for solving this problem are the microorganisms that can meet some worldwide feeding needs. Possibly the most important potential use of the microorganisms is not as a whole diet, but as a vitamin protein supplement.

The search for the possibilities for obtaining protein products rich in vitamins, amino acids and micro elements for human and animal use is a current concern. An option that can be implemented for this purpose is the crop of microorganisms, for example, using the whey or carbohydrate rich liquids, as a crop medium. In this way the composition of the whey that is rich in basic elements necessary for the fermentation is used and in turn aids in solving the ecological problem which generates its components due to its high biological oxygen demand or biochemical oxygen demand (BOD).

In order to obtain this product it is necessary to choose a microorganism using the carbohydrates present, for example, lactose (whey main component) as a carbon source for its growth. The most frequent choice in these cases are yeasts for their composition and characteristics.

The growth of the world population and the best diets derived from the prosperity in the world and in particular of China and India put in check the planet: it is estimated that for 2050, the crops and food which are produced today will be doubled.

In only 150 years, from 1900 to 2050, Mexico's population went from 11. 3 to 175 million; the world population will pass from 2000 to 9000 million, increasing another 2 billion to the current 7.

The agricultural and livestock are the main cause of the contamination of the planet, rather than that from the factories and automobiles together. Until now we believe the huge costs that face the humanity in their purpose of producing food: global warming, emission of greenhouse gases, high consumption of the water reserves, loss of biodiversity and contamination of rivers and lakes by leaks.

The current production model must be shaped so to prevent disordered growth that do will lead to ecocide.

The need to duplicate the food to feed the humanity requires changing the food paradigm and respect to the environment.

Of the 7 billion world population, 34%, 2.4 billion, is poor and the major part is suffering from food poverty.

In Mexico, the cost of the food is $45.00 per day per person. In such a way, a family of four members, monthly spends $5,400.00 only in food. National Institute of Statistic and Geography (INEGI/ENIEH) inquiry for year 2012, establishes monthly income per family:
Decil I: $2,332
Decil II: $3,931
Decil III: $5,244

Which indicates that Decyl I and II omit 1 or 2 foods and Decyl III all of the income is designated for food. In other words, 35 million Mexicans struggle daily with hunger.

Changing the production mode (use of biotechnology) and achieving producing abundant highly nutritive foods, of excellent quality and low price, appears to be a good alternative.

The traditional production mode does not aid the purpose of feeding the humanity and will continue to be the main problem of world contamination, a good part of the food industry, generates residues which contain a high level of carbohydrates and pours that organic waste into the drain and cause a strong contamination to the environment. Such waste generates unpleasant odors, causing serious diseases and damages the fertility of the soil and the quality of the water.

According with Nielsen's report on 2015, demand for high protein products will continue to grow, to date the market reports that a third party of the buyers actively search for high protein products.

One of the macronutrients of more difficult access to the diet are the proteins (it is the most expensive element of the feeding, since it is provided by food from animal origin and some vegetables). Industrial processes tend to replace this macro ingredient by carbohydrates and fats, which have a lower cost, thereby achieving the price of the product intended to be imitated.

The consequence is the intake of calories, in the detriment of a balanced diet.

TABLE 1
Effect of the lack of protein in infant development

| | | CONNECTIVE |
|---|---|---|
| DEVELOPMENT | INTELLIGENCE | TISSUE |
| Loss of height | Lethargy | Malformations. |
| Muscular weakness | Lack of concentration. | |
| Loss of appetite | Learning problems. | |
| Abortion | Problems in mental | |
| | development | |

FAO recommends an intake of 0.8 to 1. 2 Grams of protein per kilogram of weight.

Proteins are substances which carry out various functions in the cells of all living being. They form part of the basic structure of the tissues, maintain continuously and uniformly operating all of the chemical reactions that are necessary in the cells:
They form part of the basic structure of the tissues (muscles, tendons, skin, hair, nails, nervous system, reproductive system). They play regulatory and metabolic functions (nutrient assimilation, transport of oxygen and fat in blood, inactivation of toxic or hazardous materials).
Elements that define the identity of each living being since they are the basis of the structure of the genetic code (DNA).
They are an essential part of the immune system and systems for recognition of foreign organisms.
They help transport enzymes and nutrients to the cells.
They are vehicle for modern treatment of different diseases.

Proteins are organic macromolecules constituted of carbon, hydrogen, oxygen and nitrogen atoms, may also contain as well sulfur, phosphorus atoms and to a lesser extent iron, copper, magnesium and iodine, thereby grouping them to form chemical structures called amino acids.

Denaturation, of proteins, corresponds to the loss of the tertiary structure of the protein, a denatured protein having the same very open conformation and with a maximum interaction with the solvent, whereby a water-soluble protein when denatured is rendered insoluble in water and precipitates. Denaturation can be produced by temperature change, pH variation, and in some cases can be restored, a process called re naturalization.

The “biological value” of a protein is the set of essential amino acids, only present in the proteins of animal origin. It is defined by its ability to provide all of the essential amino acids to humans. The biological quality of a protein will be greater the more similar to its composition to those of the proteins of our body. In fact, breast milk is the pattern with which the biological value of the other proteins of the diet is compared.

Not all proteins that are ingested are digested or assimilated. The net utilization of a certain protein or net protein supply is the relationship between the nitrogen containing and which the organism retains.

In general, 40 to 60 g of protein consumption per day for a healthy adult is recommended. FAO recommends a value of 0.8 to 1. 2 per kilogram of body weight day. During the growth, pregnancy or lactation increases.
In growth the needs are double or triple that for an adult.
It depends on the state of health of the intestines, kidneys, since the degree of assimilation or losses of nitrogen through feces and urine varies
Including the biological value of the consumed proteins

TABLE 2
PROTEINS FUNCTIONS.

| Types | Examples | Localization of function |
|---|---|---|
| Enzymes | Fatty acid | Catalyzes the synthesis of |
| | synthetase | fatty acids |
| Reservation | Ovalbumin | Egg White |
| Conveyors | Hemoglobin | It carries oxygen into the |
| | | blood |
| Protective in blood | Antibodies | They block foreign substances |
| Homones | Insulin | Regulates glucose metabolism |
| Structural | Collagen | Tendons, cartilage, hairs |
| Contractile | Myosin | Constituents of muscle fibers |

Amino acids are the elemental units that constitute the molecules so called proteins, being crystalline substances almost every time of sweet flavor, characterized for possessing an amino group (—NH2) and one carboxyl (—COOH).

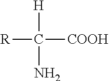

The amino acids are classified as essential and non essential, there are 20, 8 essentials for the adult and 9 for the infant.

When a protein contains the essential amino acids, it is referred to as a high biological value.

TABLE 3
ESSENTIAL AMINO ACIDS AND NON-ESSENTIAL.

| Essential. | Non - essential |
|---|---|
| Isoleucine | Alanine |
| Leucine. | Tyrosine |
| Lysine | Aspartate |
| Methionine. | Cysteine |
| Phenylalanine. | Glutamate |
| Threonine. | Glutamine |
| Tryptophan. | Glycine |
| Valine. | Proline |
| Histidine. | Serine |
| Arginine. | Asparagine |
| Taurine (felines). | |

In the last 200 years, and even today, the application of yeasts in alcoholic fermentation and bread industry is well known. These microorganisms are an inexhaustible source of new nutritional ingredients and additives of excellent functional and nutritional properties. Actually, has now increased its relevance, with the use of innovative manufacturing and fractionation techniques mainly provided by biotechnology.

Unicellular protein is the microbial biomass generated by bacteria, yeasts or filamentous fungi, grown under controlled fermentation conditions which optimize the use of different enriched or non enriched substrates.

Unicellular protein is produced from microbial biomass and in our case an yeast was selected to have the following advantages:

1. Rapid growth.

2. Simple and inexpensive culture medium.

3. Simple processing system.

4. Non pathogenic.

5. Low level of nucleic acids.

6. High nutritional value.

7. Protein concentration of 40 to 55%.

8. Easier to separate.

The essential amino acid profile is a basic factor in evaluating a protein for human consumption. In the case of yeasts, the concentration of essential amino acids such as lysine, tryptophan and cysteine are satisfactory, however, they are low in sulfurized amino acids content as methionine and cysteine, which is solved in the concentration process.

TABLE 3
COMPARATIVE EXAMPLE OF THE CONTENT
OF AMINO ACIDS OF VARIOUS PRODUCTS

| | | | | | | | | | MET + |
|---|---|---|---|---|---|---|---|---|---|
| Sources. | LEU | LIS | SER | TREO | VAL | ILE | TRI | MET | CIST |
| DESCREMED | 3.41 | 2.70 | 2.10 | 1.60 | 2.40 | 2.20 | 0.49 | 0.86 | 1.37 |
| POWDER | | | | | | | | | |
| MILK | | | | | | | | | |
| EGG | 9.00 | 6.70 | 7.70 | 5.30 | 7.20 | 5.80 | — | — | — |
| WHEAT | 6.40 | 2.70 | 4.80 | 2.90 | 4.30 | 3.80 | — | — | — |
| FAO | 7.00 | 5.50 | 4.00 | 4.60 | 5.00 | 4.00 | 1.00 | — | 3.50 |
| PATTERN | | | | | | | | | |
| (2002) | | | | | | | | | |

Traditionally the unicellular protein has been used in the elaboration of products for animal consumption, with great success. Even in the formulation of substitute milks for the growth and fattening of cattle. The FDA allows its application in foods such as flavorings, enhancers and meat extenders. However, the present development allows the application in various food systems since the content of nucleic acids is very low given the technology applied for protein purification.

Yeasts are the most widely known microorganisms, best studied and generally better accepted by consumers. The yeasts are rarely toxic or pathogenic and can be used in the human feed. Although the protein content does not exceed 60%, its concentration of essential amino acids such as lysine, tryptophan and threonine is satisfactory, although its methionine and cysteine content is low. The yeasts are very rich in vitamins (group B) and their content in nucleic acids is low as it is in the range of 4 to 10%.

Yeasts have been defined as microscopic, unicellular fungi, most are multiplied by budding and some by excision. This group of microorganisms comprises about 60 genera and about 500 species. Historically, studies on enological microbiology have focused on yeasts belonging to the Saccharomyces genera, which are responsible for alcoholic fermentation. It was previously believed that only them were involved in the alcohol production process, however, the different non—Saccharomyces yeasts, especially during the initial phase of the fermentation, can influence the organoleptic properties of alcoholic beverages.

The role of yeasts as fermenting agents was not recognized until 1856 by Luis Pasteur. Yeasts are the agents of fermentation, there are a large number of species that differ in their appearance, their properties, their forms of reproduction and the way they transform sugar. Wine yeasts belong to several genera, each divided into species. The most widespread species are Saccharomyces ellipsoideus, Kloeckera apiculata and Hanseniaspora uvarum, which alone represent 90% of the yeasts used for wine fermentation. Like all living things, they have precise needs in terms of nutrition and the environment in which they live. They are very sensitive to temperature, they need an appropriate diet rich in sugars, mineral elements and nitrogen substances, they have short reproductive cycles, which makes the beginning of fermentation so fast, but, as they multiply, they can die from lack or excess of the mentioned variables.

The document Taron-Dunoyer A, Pérez-Mendoza J, Martinez-Zambrano J. “Obtaining unicellular protein from whey”, Vitae volume 19, number 1, January-April 2012, describes a process to obtain unicellular protein a from whey, where 250 ml of whey plus 7 ml of 10% trichloroacetic acid is used as culture medium, precipitating the casein by heating, clarifying and sterilizing the whey, the biomass is centrifuged, the filtrate is discarded and the biomass protein is dried at 30° C. for 24 hours, obtaining a consumption of 68% lactose in the medium.

In this document no enzymes are used, since the starting raw material (whey) is the preferred means of producing unicellular protein by the yeast Kluyveromyces lactis. With respect to what is indicated in this document, it is necessary that the strain used is different from that used in the invention described in the present application (Kuyveromyces marxianus ours strain) vs Kuyveromyces lactis used in the process described in the document, at the same time it is indicated that In the claimed process you can use unprotected serum or not, the culture medium is not sterilized, the nutrient mixtures are different, in our process there is no flocculation, and the final product in our case we obtain intracellular protein, and no yeast.

The document Marta Elena Cori de Mendoza, et. al “Obtaining and characterizing two protein concentrates from biomass of Kluyveromyces marxianus var. Marxianus grown in deproteinized whey”. Scientific Magazine vol. 16 No. 3 of 2006, describes a process where deproteinized and supplemented lactoserum is used, to adjust lactose to 1.5% by supplementing with ammonium sulfate, the yeast used is Kluyveromyces marxianus, fermentation is performed using a silicone-based antifoam, The document indicates that numerous substrates for the production of unicellular biomass have been studied, with milk whey being one of those that has been considered most important due to its low price and availability, apart from the fact that it is usually an important contaminant in wastewater, which causes serious problems in the operation of the purification plants.

Therefore, it is proposed to use as a fermentation substrate for microorganisms capable of assimilating lactose, such as Kluyveromyces marxianus var. Marxianus, with the purpose of obtaining microbial biomass, also known as “unicellular protein”. This has a protein content that ranges between 40 and 80% on a dry basis and its quality is more similar to animal protein than vegetable. However, the use of unicellular protein for human consumption has important limitations: the presence of cell walls, since these reduce the bioavailability of proteins, also contain antigenic and allergenic factors; the high content of nucleic acids (basically RNA-->ARN) which can cause kidney disorders and gout, and finally the reduced functional properties exhibited by dehydrated cell biomass. These limitations can be overcome by the preparation of protein concentrates, as described for Candida tropicalis and for Kluyveromyces marxianus var. Marxianus, using alkaline extraction and isoelectric precipitation; However, this is not enough, since the levels of nucleic acids must be reduced, either by chemical methods or enzymatic methods. Among the chemical methods, one of the most accepted is phosphorylation with phosphorus oxychloride or alternatively with sodium trimetaphosphate, where protein concentrates have been obtained from Saccharomyces cerevisiae, which have then been dehydrated through sophisticated methods, such as freeze drying or spray drying, respectively. The results described in the document demonstrate that a protein concentrate can be obtained from microbial biomass Kluyveromyces marxianus var. marxianus grown in deproteinized whey. The yield and cell concentration values obtained were satisfactory for this microorganism.

Although what is described is a process more similar to the protection object of the present application we can point the following differences: in our process we do not use Complex B (vitamins), additionally the medium is not sterilized, nor carries three heat treatments, although it is an alkaline extraction, in our case there are three processes involving enzymes (cellulases and proteases), the protein obtained being soluble whereby the application is varied, since it does not have a taste or color that prevents its application in dairy or over the entire range of the food industry, on the other hand after the isoelectric precipitate the protein is diafiltered and concentrated by ultrafiltration, raising its protein content to 80% dry basis, in this case it is part of a powder in powder, in our case using liquid serum either sweet or acid, the acidifying medium is acetic acid in our case using phosphoric acid or any organic or inorganic acid, the extraction temperatures differ, is not only 40° C. but we have three treatments with temperatures of 68° C., addition of enzymes, pH modifications both acidic and alkaline, the protein is not milled in blender and spray dried.

The Bird G, et al. document “Amino acid profile of the unicellular Protein of Kluyveromuces marxianus var Marxianus”, Interscience Vol. 33, No. 44, 2008, Discloses that “ the yeasts of Kluyveromyces, canida, Schwanniomyces, Lipomyces, Pichia, Rhodotorula and saccaromycosis are ideal for the production of Unicellular protein (PUC). They are characterized by having a weak fermentative metabolism, do not have a glucose effect and grow on different substrates: molasses, lactoserum (Kluyveromyces), methanol (Pichia), acid hydrolysate of sugar cane bagacalins (Saccharomyces cerevisiae and Utius Utility), amylaceous waste water (Shwanniomyces), sulfate liquor of the paper industry, or n Alkanes (chokes). The document discloses that when the substrate is whey and molasses, the yeast to be chosen Is Kluyversoucis marxianus var marxianus, which is checked by determining that the amino acid profile obtained in the stated document shows a balanced distribution of the content of these in the unicellular protein of K. marxianus, as compared to the international (FAO) reference standards and egg protein and other conventional protein sources, suggesting the potential use of this unicellular protein (PUC) as a protein source. While the document has the amino acid profile, the method of producing the yeast is standard and differs entirely with our process in the following way; in our case it can be used to use deproteinated serum or not the operating temperatures differ, us at 38° C. and at pH 4.2, the medium is not diluted, did not sterilize the culture medium, we used the culture medium, we used fresh or acidic serum and obtain the intracellular protein and not the yeast, in a second treatment, finally it can be clarified that in this process phenylisocanate is used to extract the amino acids and then enter columns, which makes it invisible to human consumption.

The Spanish patent document No. ES 2 050 066, published Jan. 5, 1994, discloses a method consisting of three differentiated stages:

1) the invention relates to The treatment of the whey. Consisting essentially of a previous deproteinization, followed by the addition of a number of nutrients necessary for the fermentation (mainly a nitrogen source, a source of phosphorus, mineral elements and vitamins) which have been studied and fixed to their levels in order to obtain an optimum culture medium for the fermentation process. Thus, the delivery of growth factors and vitamins, resolved into other systems through the addition of yeast extract, high cost and causing the formation of foams, is substituted in this invention by the use of a vitamin complex of composition and pre optimized concentrations.

In this way, a covering of the process is achieved, while the formation of foams is decreased. The fermentation medium is finally subjected to a pasteurization process prior to fermentation.

2) Fermentation. In this step the working fermenter is inoculated from a previous culture. The fermentor is continuously aerated and controlled by the variables, temperature and aeration, as well as the formation of foams, the whole of this step being developed under sterile conditions.

3) Separating/collecting the product. The protein rich yeast is separated from the effluent by continuous centrifugation. The effluent can be poured with minimal contamination problems. The yeast cream is stored in tank until ground, dried and collected as final product.

The differences in the process object of the present patent application are: in our case it can be used to use deproteinated serum or not, the operating temperatures differ, we at 38° C. and at pH 4.2, the medium is not diluted, the medium was not diluted, using the culture medium, using sweet or acid serum and we obtain the intracellular protein and not the yeast, in a second treatment.

Ghaly, A, M Kamal and L. Correia 2005, document. “Kinetic modeling of continuous submerged fermentation of cheese wheat for single cell Protein Production”, bioreource Technology 96 (2005), 1 143-1152, discloses the fermentation of cheese whey for the production of unicellular protein using the yeast K. fragilis as a biochemical reaction of cells and lactose to produce cells as the main product.

The differences in our process with respect to that described in the document, is that we can use deproteinated serum or not, the operating temperatures differ, we at 38° C. and at pH 4.2, the medium is not diluted, not sterilising the culture medium, fresh or acid serum and a different strain are used.

The Chinappi Italy and Sanchez Crispín José A. 2000, document. “Production of Kluyvertoworms fragilis biomass in deproteinized milk serum”, Acta Cientifica Venezolana, 51: 223-230, describes the results of a study performed with the whey from a plant of aged cheeses, deproteinized by acid thermocoagulation (pH 4.5 and 90/−EC) and supplemented with ammonium sulfate (1 g/l), which is a good culture medium for producing, by directed aerobic fermentation, biomass of the yeast Kluyvertoworms fragilis. Optimum experimental conditions were established to obtain the maximum production of biomass in fermentors of different design and capacity. For lactose concentration of 15 g/l, pH 4.5, 30/−EC and aeration between 0.25 and 1 VVM, the doubling time was less than 2 hours and 98% of the lactose was consumed. Under these conditions a dry weight yield between 36 and 49% (g biomass/g lactose) was obtained. The biomass (unbroken cells) contains 46% proteins on dry basis and exhibits a “in vitro” digestibility of 65%. The organic charge of the medium decreased 80% after 12 hours of fermentation. The invention relates to a method for eliminating a contaminating waste and, simultaneously, producing an insulin which may have industrial interest as a protein supplement in animal concentrates for animals. The technology applied to this work uses the wastewater waste, supplemented with low concentrations of yeast extract, as a culture medium for the production of biomass of the yeast K. fragilis with high protein and vitamin content and adequate digestibility. This biomass can be used as a nutritional supply in diets intended for animal feeding. Simultaneously, the fermentative process removes more than 80% of the organic loading of the whey from the milk, avoiding what contributes to increasing environmental contamination.

The document describes a process that part of a fresh liquid serum which is deproteinized, the first difference being that the invention can be deproteinized or not, and can be acidic or sweet, a biomass is obtained which is then dried and then subjected to an enzymatic treatment with pepsin, which is the main difference since our process allows obtaining the protein from the cell, without drying and using cellulase and proteases, the protein being purified using acetone and then washed in our case it is subjected to isoelectric point and then purified by diafiltration in a tangential filtration system (Ultrafiltration), the serum is diluted to adjust its lactose content to 2%, and in this case this is not the whole serum part and is decream, no vitamins are added and the nutritional supplement system is different.

The Zumbado-Rivero Wendy, Esquivel-Rodríguez Patricia, Wong-Gonzalez Eric, 2006 document, “Selection of an yeast for the production of biomass: growth in cheese serum”, Mesoamerican agronomy, 17 (2): 151-160, describes a study for the selection of an yeast for the plant production of biomass: growth in cheese serum. The study was performed using the milk whey of the Turrialba white cheese making process. The Species Kluyveromyces marxianus, canido kefyr and Saccharomyces cerevisiae were compared by their growth in a batch fermentation system, the fermentation time determined the total productivity and protein content of the biomass in a red study that the yeast species Kluyveromyces marxianus is the best option for the production of unicellular protein, for presenting a lower fermentation time, greater productivity and equal protein content of the biomass than the other yeasts, in addition to utilization facilities.

In this case, the document relates to the identification of specific yeasts for the optimization of the production of biomass on defined substrates, the aforementioned processes are general processes for producing biomass, more non specific, interfering with our process at temperatures and nutrients, moreover, from biomass and extracting the protein, which factor is not considered in the document, without breaking the cell wall.

The Buitago Glorymar, et al, 2008, document, “Continuous production of single cell protein of Kluyveromyces marxianus var marxianus”, Tech Magzyne. Zylia University, 31document, (Maracaibo Special), describes the effect of lactose concentration on the growth kinetics of Kluyveromyces marxianus var marxianus ATCC 8554 and the production of b-D-galactosidase enzyme in previously deproteinized whey, the document notes that in the production of unicellular protein for human and animal consumption, microorganisms are Used as: Saccharomyces Cerevisiae, Candida utius, Fusarium and Kluyveromyces marxianus, the latter is a microorganism which rapidly fermentates the lactose, sugar which constitutes the essential part of the dry extract of the dry extract of the serum at a concentration of 4 to 5% p/v. The study showed that K. marxianus biomass can be obtained at low concentrations of lactose in 50% yields, which generates the possibility of establishing a continuous production system of unicellular protein for use as a protein concentrate.

The Degrossi Claaudia and Wachsman Monica, 2004, document, “Study of some characteristics of the yeast strains and of their cell yield using a whey based culture medium”, University of Belgrano, Faculty of Exact and Natural Sciences, Degrre in Pharmacy thesis. In this work the cell yield of different strains of yeasts was evaluated using the whey as a culture medium. At a first time, the microscopic and macroscopic characteristics of the yeast strains Were analyzed (Kluyveromyces fragilis Mp-8, Saccharomyces cerevisiae Mp-12, Canido humicola Mp-6), then the ability to use the lactose was verified as a carbon source, the literature data was confirmed in relation to the ability of these strains of using the lactose. The presence of proteases (by the method of fluidization of gelatin) and amylases (by iodine staining), which could allow for the replacement of the source of the components of the culture medium (for example, the sub-products of the alcoholic beverage industry) can also be used. The cell yields of the strains were compared separately and in whole, using as a culture medium the dairy whey. The tendency of the binary cultures to exhibit a higher cell yield was observed.

The difference in the state of the art and the process described herein is that in our case we can use deproteinated serum or not, the operating temperatures differ, the medium is not diluted, the culture medium is not sterilized, fresh or acid serum can be used and we obtain the intracellular protein and not the yeast, in a second treatment.

The Araujo Karelen, Páez Gisela, et al et al, document, “Effect of lactose concentration on the growth kinetics of Kluyveromyces marxianus var marxianus and the production of beta galactosidase”, the effect of lactose concentration on the growth kinetics of Kluyveromyces marxianus var marxianus ATCC 8554 And the production of the Enzyme Beta D-galactosidase in previously deproteinized whey were studied.

The differences regarding this document of the state of the art is that in our process deproteinated serum may be used, the operating temperatures differ, the medium is not diluted, the culture medium is not diluted, we obtain the intracellular protein and not the yeast, in a second treatment and does not utilize lactase.

### BRIEF DESCRIPTION OF THE FIGURES

FIGS. 1-4 are flow charts depicting the steps in accordance with the present invention.

### DETAILED DESCRIPTION OF THE INVENTION

The invention contained in the present patent application offers a solution for optimum use of food, as well as the reduction of pollution associated with the waste of nutrients that are discharged into drainage and the environment. The invention is based on sustainability and can be replicated in different parts of the country, since whey, molasses, cane bagasse can be used.

The whey of milk is the result of coagulation of the production of cheese, either by the use of renin or coagulants or acidifying. Yellow green colored, opaque and contains between 6.0 and 6.5% solids. This constitutes about 50% of the solids of the milk.

There are different varieties of serum and the basic classification can be considered as sweet sera those derived from milks with acidity of between 14 and 16° D, with a natural acidity of between 9 and 11° D (the most valuable of all) pH 5.8-5.6, acid with acidity greater than 16° D pH 4.0 to 5.8, generally from directly acidified cheeses or of cheeses which are allowed to ripened before cutting the curd and finally the salted sera containing a high level of salt from the salting processes.

There are different treatments for the serum comprising from their use as a liquid for the manufacture of beverages, dairy foods, dairy beverages to the production of high commercial value enzymes and amino acids and the production of water.

The serum is a rich source of valuable nutrients and can tell that through different process steps it can be recovered for human feeding.

The production of a unicellular protein has been encouraged since the 1980's, as a response to the constant increase in the world population.

In general, it has been determined that the protein content in yeast is from 45 to 55%, with the added value of containing a low level of RNA, thereby enabling its application as a food supplement, both in cattle feed and human consumption.

The invention contained in the present patent application is a process for fermenting lactose and molasses, for obtaining a single cell biomass, the process for obtaining a high protein value product which will be in the range of 50 to 90% for application in the food industry. The propagation means may be prepared from whey or molasses milk whey. In the case of the embodiment of the process where serum is used that will contain all of the remaining ingredients, specifically lactose, carbon source for use by Kluyveromyces marxianus.

The process allows the substrate to be obtained from sweet whey, acid, or any fermentable sugar liquid or high water in starches. The whey and high water leached concentrates represent a serious source of contamination, in general with an oxygen biological demand equivalent to 60,000 mg/L.

The BOD (biochemical oxygen demand) which measures contamination, indicates that, for each liter of serum discarded, an equivalent contamination is produced that produces 0.68 people. And in spite of which in some cases the serum is already given an added value, in recovering the protein therefrom, the problem of which to make the permeate and the acid serum is still remaining.

The present invention adds value to a product which in its case does not represent a strong ingress and benefits to the environment in its application, using simple systems present in the operation of a food and fermentation plant, allowing the use of high residual sugar or carbohydrates.

During the process specifically in the fermentation there are parameters for measuring, allowing the process to be defined, the main parameter being the production of biomass at a specific time. Due to the importance in the development of the biomass, a strain is chosen that is capable of growing in short time. Thus, defining the amount of protein to be produced and hence the corresponding economic factors.

Although commercial yeasts exist for making the fermentations, it is more effective to use pure yeast cultures that proceed from the area where they are to be used, which are known as selected local yeasts, since it is believed that the yeasts found in a micro-zone are: specific of the area, fully adapted to the climatic conditions of the zone and to the raw material, that is to say, the must to be fermented, are at least partially responsible of the unique characteristics of the products obtained.

The inherent characteristics of the zone can therefore be an interesting aspect to the time of selecting a yeast, although there are many others that are also to be taken into account. The importance of these parameters can be relative, depending on the product for which they want to be used.

Thus, selection of the appropriate strain for each type of fermentation is a very important strategy for ensuring on the one side a correct fermentation, as well as to improve the characteristics of the final product, as the yeasts can produce compounds which give a distinction to the product obtained, such as glycerol, esters, higher alcohols, etc.

In evaluating the yeast K. marxianus for biotechnological applications, it is impossible to not consider other more popular species, Mainly S. cerevisiae and K. lactis. The first is probably the most widely used biocatalyst in the biotechnology industry and a model organism in biological studies, while the second has been chosen as a negative Crabtree model. The fact that several strains have obtained the general regarded as safe (GRAS), similar To S. cerevisiae and K. lactis indicates that this aspect has no disadvantage for the former, as compared to the latter yeasts, in terms of the process of approval of regulatory agencies. The fact that K. lactis was chosen by the scientific community as a model organism in the Kluyveromyces genus, and Not K. marxianus led not only to a better understanding of its physiology, and for the complete sequencing of its genome, but also for the development of various applications, including the expression of more than 40 meteorological proteins. This is due, in large extent, to the fact that researchers have used, since the principle, a very small number of isolated K. lactis that has not been the case of K. marxianus species.

The unicellular protein is produced from microbial biomass and in our case is selected From Kluyveromyces marxianus (yeast) by presenting the following advantages:

1. Rapid growth.

2. Simple and inexpensive culture medium.

3. Simple processing system.

4. Non pathogenic.

5. Low level of nucleic acids.

6. High nutritional value.

7. Protein concentration of 40 to 55%.

8. Easier to separate.

Accordingly, the yeast Kluyveromyces was selected marxianus (K m) for its capability of high reproduction, production of biomass and fermentation of carbohydrates, the production of biomass corresponds to 0.3 g/l/H at a time frame of 20 hours and a protein content of 32%.

The variations in the substrate used for the growth of the yeast and their fermentation and refining conditions will define the composition of the same the invention relates to a final product.

The protein obtained generally contains 80% amino acids, 12% acids nucleic acids and 8% of ammonium, in the present invention are eliminated. tangential filtration. The digestibility and biological value are high, being found between 85 and 97% depending on the purification processes, highly valued from the point of biological view.

A wide variety of culture media exist for growing the micro milk organisms, in fact, any medium containing lactose, water and minerals, it is responsible for the development of these microorganisms.

The process starts with the obtainment of Kluyveromyces marxianus, content in a glycerol at −45° C. or in a strain propagated to an environment in the laboratory, this glycerol is introduced into a pre sterilized and adjusted medium for the correct propagation of the strain. The fermentation culture medium is prepared according to the following formulation:

| Description | Amount in % |
|---|---|
| Carbohydrate (lactose o molasses) | 4.50 |
| Ammonium sulphate | 0.60 |
| Magnesium sulphate | 0.20 |
| Yeast extract | 0.60 |
| Potassium Mono Phosphate | 0.60 |
| Urea | 0.40 |
| water | 93.10 |
| Total | 100.00 |

The medium is prepared, the pH is adjusted to 4.0 with a solution of concentrated phosphoric acid or other organic or inorganic acid; sterilized at 250° C. 15 psi pressure, during 30 minutes.

The pH is removed for cooling and the pH is reset to 3.8, to cool to 38° C. and in a sterile laminar hood, the whole glycerol is transferred or the culture prepared to allow its reproduction.

This is incubated in an orbital incubator during 12 hours, 150 rpm 38° C. being scaled, following this procedure until reaching fermentation volumes of 10 and 30 liters, to continue the scaling in fermenters of capacity of 10 to 50 liters, in order to achieve an inoculum at an industrial level equivalent to 1% of the total volume of the volume to be processed at the industrial level.

During the laboratory fermentation processes, foaming must be controlled, adding a silicone based antifoam, the pH is adjusted using phosphoric or organic or inorganic acid or NaOH or KOH diluted to 10%; the temperature should not exceed 40° C. nor decrease from 36° C. a 1 22 L of air per liter of culture medium is added per hour and the oxygen dissolved in saturation will be searched; having passes a period of between 12 and 20 hours according to the graph of oxygen consumption and optical density. The biomass is harvested by means of a high capacity centrifugal separator; for the process, separator can be considered separator by nozzles or the application of membranes.

The clarified by containing lactose should be fermented using the same procedure up to three times in order to achieve the total consumption of the carbohydrate.

Once the biomass has been obtained, it will be diluted in a drinking water solution to 10% total solids content and the first enzymatic treatment will proceed according to the following description:

(Note the waste water will be subjected to a membrane filtering process and electrodialysis for the recovery of drinking water).

Biomass diluted to 10%.

The pH is adjusted to 6.8 with phosphoric acid. It is heated to 68° C. for 60 minutes at 120 rpm.

The temperature is adjusted to 80° C. and the pH to 9.0 with sodium hydroxide solution or 1% potassium hydroxide.

Addition of basic cellulase enzyme 1 g per Kg of biomass, maintaining the conditions for 15 minutes. After the period of time, the biomass is separated, either by centrifugation or by membranes. The clarified liquid is stored and the debris (biomass) is subjected to a second enzymatic treatment; as a first step the pH is adjusted to 6.8 with a phosphoric acid and heated to 68° C. maintained for 60 minutes.

The pH is adjusted to 9.0 with sodium hydroxide or potassium hydroxide and the alkaline cellulase and the granular cellulase are again added per kg of biomass obtained respectively, maintained at 80° C. during 15 min.

The debris is again separated by centrifugation or membranes and the clarified liquid is saved.

The waste is exposed to a third enzymatic treatment according to the following procedure: the pH is adjusted to 6.8 with phosphoric acid and allowed to warm to 68° C. maintained durance 60 minutes.

The pH is adjusted to 9.0 with sodium hydroxide or sodium hydroxide and the protease is added at a ratio of 1 g per kg of biomass obtained, maintained at 80° C. for 15 min.

The debris is again separated by centrifugation or membranes and the clarified liquid is saved.

The debris is saved for later use.

The clarified extracts are brought together and the pH is adjusted to 7.0 with basic solution and concentrated to concentrate by membranes and evaporation so as to eliminate the majority of the salt content, sugars and to increase its concentration at 30 and 60%, respectively prior to drying.

The product is subjected to drying at a temperature of 140° C. entrance to dryer and 71° C. to the dryer outlet.

The yield is 0.83 g of protein per gram of carbohydrate.

The fermentation medium used is: ammonium sulfate (0.15%)+magnesium sulfate (0.05%)+yeast (0.15%)+monopotassium phosphate (0.15%)+urea (0.1%)+water+carbohydrate (lactose and molasses) (4.5%). Adjust to pH=4 with phosphoric acid. It is pasteurized at 85° C. the pH=3.8 is incubated for 12 to 20 hours at 150 rpm at 38° C. a foam controller is added with silicone and pH regulation with phosphoric acid or 1% NaOH, temperature of 36 to 40° C.±1. The invention relates to an air/L L of culture medium.

The biomass is centrifuged and washed with water and 1 g/l sodium hexametaphosphate (to remove excess sugars), the biomass diluted to 10% of total solids with water. This is subjected to the first enzymatic process, adjusting pH to 6.8 with dilute acid, heated to 68° C./60 min at 120 rpm. Adjust temperature to 80° C. and pH 9.0 with 1% NaOH solution adds the cellulase enzyme 1 g/kg of biomass keeping the conditions for 15 min the clarified liquid is centrifuged or separated with membranes, the clarified liquid is stored and the biomass is subjected to a second enzymatic treatment: adjusting pH to 6.8 with acid and heated to 68° C./60 min. adjust pH to 9 with granular cellulase NaOH and alkaline cellulase 1 g/kg biomass at 80° C./15 min; is centrifuged or by the process of membranes, the clarified liquid is saved and the biomass obtained is subjected to an enzymatic treatment: adjusting pH to 6.8 with dilute acid, heated to 68° C./60 min at 120 rpm. adjust temperature to 80° C. and pH 9.0 with 1% NaOH solution. Add the protease enzyme 1 g/kg biomass at 80° C./15 min. it is centrifuged, the liquid is saved and the biomass is discarded.

The protein heats are brought together and the pH is adjusted to 7 with basic solution, concentrated by membranes and evaporation, where the content of salts and sugars is removed, concentrated at 30 and 60% before drying. It is dried at 140° C. inlet and 71° C. exit in dryer.

Yield of 0.12% protein/g Carbohydrate.

More specifically, the process comprises the steps of:

1. Receiving serum:

Once the product is released, it is connected to the discharge line, through the Tygon hose, connecting to the deaerating discharge pump.

2. Send to the cooling press where the temperature is decreased to 4° C.

3. The product will be maintained in the silos at 4° C. with constant agitation.

4. It is discharged through the bank of the corresponding valves and the pump to be sent to the balance tank of the pasteurizer, where it is cooked and brought to 85° C./15 seconds.

5. send to the balance tank of ultrafiltration membranes.

6. The permeate is sent to storage tanks. The nutrients will be added in 50% w/V solution via the feed line to storage tanks.

The ratio of nutrients to be added will be according to the following table:

Nutrients to be added per gram of lactose

| Name | amount. |
|---|---|
| Lactose | 1.00 |
| Ammonium sulphate | 0.15 |
| Magnesium sulphate | 0.05 |
| Yeast extract | 0.15 |
| Potassium Mono Phosphate | 0.15 |
| Urea | 0.1 |
| water | 98.4 |
| Total. | 100.00 |

7. From the storage tanks it is sent to a tank through the corresponding pump and the bank of valves to the balance tank of the plate exchanger to bring the product to 85° C./1 min and lower the temperature to 40° C. sending to the fermenters.

8. The fermenter which has previously been washed and sterilized with sanitary steam in the event of following the batch fermentation process, in the case of a continuous process will continue according to the needs of the process.

At this stage the volume of air to be injected should be controlled to be maintained in a ratio of 1. 5 Liters of air per liter of sugar dilution, the pH range 5.6 to 4. 6, the agitation will be maintained at 200 rpm, the temperature will be in the range of 39 to 41° C., the foam generation must be controlled automatically through the silicon dioxide metering, the medium will be acidified with a phosphoric acid solution and basified with a 50% sodium hydroxide solution.

In the case of batch processing it will be fermented for 20 hours, if there is continuous fermentation the replacement rate will be 20%.

As part of the control, it should be counted with the graphical representation of: oxygen consumption, pH variation and temperature as well as with the growth curve.

9. The product is pumped into the decanter or centrifuge by nozzles where the product will separate into biomass and clarified.

10. The clarified is sent to the storage tank where it will be pumped to the balance tub, the equivalent to 5% of the production volume, the remainder being stored for the latter treatment of nanofiltration and reverse osmosis in order to recover water for use in services and dilution of the enzyme treatments. The treated will be stored for later use (permeate) and the residual will be discarded (retained).

11. The resulting purified biomass is pumped to a lung tank to be diluted by a solution of water buffered with sodium hexametaphosphate (1 g/per liter) in order to remove carbohydrates. The final solids concentration should reach 10%.

11A. The solution is pumped to be separated by decantation or by centrifugal nozzles, wherein the biomass is pumped into the first enzymatic treatment and clarified by the treatment of nanofiltration and reverse osmosis.

11B. In the enzymatic treatment tank, the biomass is diluted to 10% of total solids, passing through a plate exchanger to raise the temperature to 68° C.

12. Once the dilution is achieved, the pH is raised to 6.8 keeping it for one hour. This being done by a heat exchanger plates with the corresponding pump for raising temperature to 80° C., adjusting the pH to 8.0 with the addition of sodium hydroxide. Adding as well 1.5 g of PG—02 (Granular Neutral Cellulase) per Kg of dry base biomass dissolved in 10% water, thereby keeping it for 15 minutes.

13. The solution is pumped by pumping to separate by decantation or by centrifugal nozzles, wherein the biomass is pumped into the second enzymatic treatment and the clarified into the protected broth tank for storage, with a content of 3% of total solids.

14. In the second enzymatic treatment tank, the biomass is diluted to 10% of total solids. Passing through a plate exchanger to raise the temperature to 60° C.

15. Once the dilution is achieved, the pH is raised to 6.8 to maintain it during one hour, this is done by a pumping plate exchanger to raise the temperature to 80° C., adjusting the pH to 8.0 with the addition of sodium hydroxide. Adding as well 1.5 g of PG02 and PG04 (Alkaline protease conc.) for each Kg of dry base biomass dissolved in 10% water, maintaining it like that during 15 minutes.

16. The solution is pumped to separate by decantation or by centrifugal nozzles, wherein the biomass is sent by pumping to the second enzymatic treatment and clarified to the tank of proteinaceous broth for storage, containing 2.8% of total solids.

17. In the enzymatic treatment tank, the biomass is diluted to 10% of total solids, passing through a plate exchanger to raise the temperature to 60° C.

18. Once the dilution is achieved, the pH is decreased to 4.5 with phosphoric acid to thereby maintain it like that during one hour. Being this done it is passed to a pumping plate exchanger to raise temperature to 80° C., adjusting the pH to 8.0 with the addition of sodium hydroxide. Adding the same 1.5 g of Luco (Cellulase conc. AL2X) for each Kg of dry base biomass, maintaining it like that during 15 minutes.

19. The solution is pumped to separate by decantation or by centrifugal nozzles, where the biomass is pumped into the collection container and clarified to the tank of proteinaceous broth for storage it will contain 2.58% of total solids.

20. In the storage tank, the solids content and protein content will be monitored. The pH is adjusted to between 6.8 to 7.0, with sodium hydroxide or phosphoric acid according to the case (reaching 2.8% total solids and 52% protein). The tank must be equipped with agitation. From here it is sent to to the membrane filtration system for removing the salts and increasing the protein content.

21. It is sent by pumping to the ultrafiltration system.

22. The retained is sent to a lung tank where the protein, carbohydrate, salt and pH content will be monitored (at this stage a protein concentration of 68 to 70% and 9% solids) is reached. It immediately passes to nanofiltration wherein a protein content of 80% and 30% of total solids are reached. The next step is electrodialysis wherein a protein content of 92% is reached by maintaining the 30% solids by removing the nucleic acids at the same time, and in this step, pasteurization is performed. Once this is done, it is sent to the evaporation process reaching a solids content of 40%.

23. The concentrated product is then spray dried at an inlet temperature of 160° C. to 180° C. and at an exit temperature of between 60 to 80° C.

24. Finally, it is sawed and stored.

25. In the ultrafiltration and nanofiltration systems, the retained is pumped into a lung tank where it is maintained to pass to evaporation, in this stage it is heated by plate exchange at 60° C., the permeate is sent to the lung tank to be able to accumulate with the remainder of the clarified biomass and the biomass wash water in order to be subsequently sent to ultrafiltration and reverse osmosis.

All of the product is pasteurized prior to entry into the filtration system, the permeate is passed through ionic resin and by an electrodialysis system, to reuse the water in the system. The water of the retentate is discarded.

The final product can be used as a food supplement of white bread, sweet bread, dairy formulas, fruit beverages, added tortillas, etc.

### BRIEF EXPLANATION OF THE FIGURES

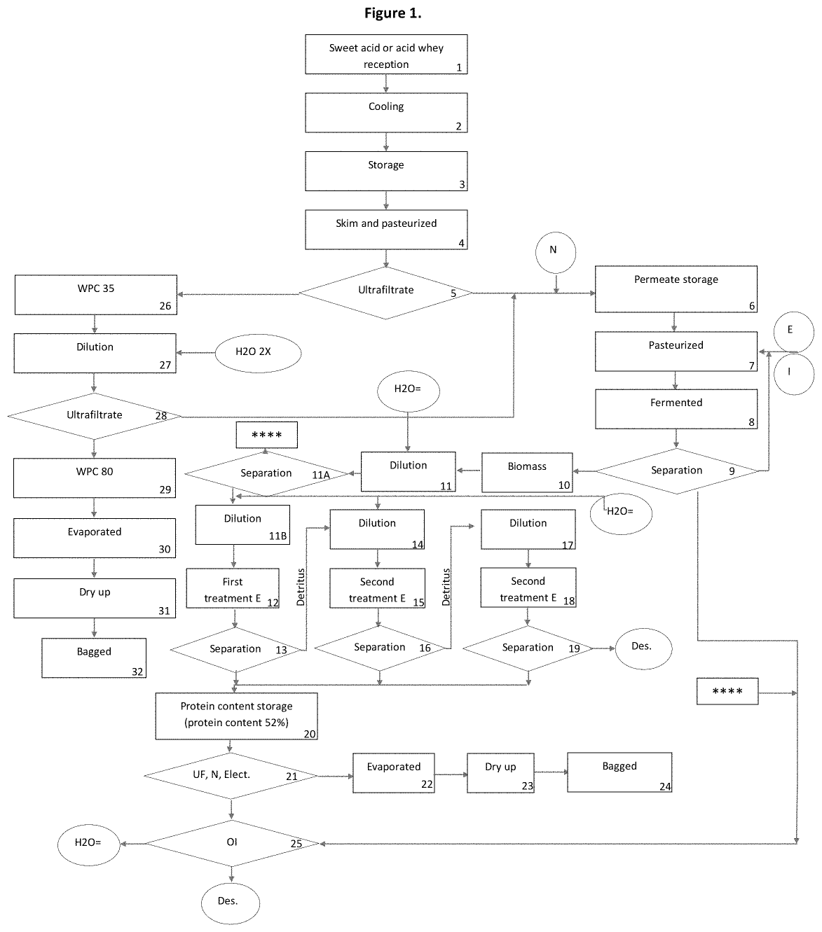

In one embodiment of the process (FIG. 1) this begins with the step of receiving the serum:

The process has the following steps:

1. Reception of Sweet or Acid Whey:

The serum is received by pipe or by pipe directly, in case it is through pipes, this upon arrival must be weighed and passed to the reception area, once it is in the reception area it will proceed to stir by spaced 10 minutes and a sample will be taken.

Once the 500 ml sample is taken, the following parameters are determined:

| Parameters. | |
|---|---|
| Temperature | 6 an 8° C. |
| acidity | 14 a 16° D |
| Lactose | 4.5 a 5.0% |
| Density | 1.018 a 1.022 Kg/L |
| Totals solids | 6.0 a 6.05% |
| Protein | 0.72 a 0.78% |
| Antibiotics | Negative |
| Inhibitors | Negative |
| Chloride derivates | Negative |
| Grass | 0.15 a 0.30% |
| alcohol stability | Negative |
| Ebullition | Negative |
| Rezasurina | Good |

As part of the treatment processes for the batch of serum, an organoleptic analysis is made, for determining:

| INDI- | | | QUALITY |
|---|---|---|---|
| CATOR | SWEET SERUM | ACID SERUM | SPECIFICATIONS |
| Odor | Characteristic | Characteristic | Characteristic |
| Color | Greenish yellow | Greenish yellow | Greenish yellow |
| Flavor | Characteristic | Characteristic | Characteristic |

Additionally, the pH, density, acidity, fat content, dry matter and crude protein, and the concentration of lactose, calcium, phosphorus of 2 varieties: sweet and acid, of cheese serum will be determined.

It is worth mentioning in the case of the serum acid modality, there is a decrease in pH and lactose content, and an increase in acidity and salt content.

Once the product is released, it is connected to the discharge line, through a Tygon hose, connecting to the air discharge pump.

2. Cooling.

The obtained serum is sent to the clarification, in order to remove the suspended solids, afterwards it is sent to cool through a plate press, where the iced fluid exchange will occur, with the serum in order to decrease its temperature to at least 4° C., further in this step the control of conditions such as temperature, percentage of solid in suspension, lactose content, protein, acidity, stability, inhibitor and fat content will be made.

3. Storage.

The serum is sent to the silo tanks, which are provided with isolation of at least 4° C., where the serum is retained until use, such silos have constant agitation, man entry, CIP washing System, level and ventilation.

In this stage, level, temperature and input and/or output dates are performed.

4. Skim and Pasteurized:

The serum is discharged by means of a bank of valves and is sent to the balance tank of the pasteurizer through the pipes and the corresponding pump.

The serum is sent to be pasteurized at 80° C. for 15 seconds and at this stage the skimmer is included to eliminate the fat content to a maximum, taking it to at least 0.25%, it is sent to the lung tanks at 4° C., said tanks have agitation and must be isolated, with level indicators, man input and CIP system.

This step is a critical control point whereby corresponding controls of temperature, pressure, flow control pumps, diversion valves, suitable balance tank level, etc. are to be carried out.

4 Bis. Storage in Lung Tanks:

Once the serum is pasteurized, it may or may not be sent to the lung tanks ensuring a maximum temperature of 4° C., the lung tanks must be isolated, having agitation, level indicators, man input and CIP System.

In this step there is a risk of microbial contamination whereby the sanity and bacteriological control process become critical control parameters, further the temperature, residence time, fat content, rezasurine and acidity should be controlled.

5. Ultrafiltration:

To feed the ultrafilter (UF) it must be ensured that the fat content is at maximum 0.02% and the rezasurine is good (without change in 4 hours), if these parameters are not met, the membranes will be covered and the retainer will have a high account The UF also concentrates bacteria, so this is a critical point of bacteriological and mechanical control.

It is considered that in this process the flow per square meter of membrane is 10 liters per hour, with a maximum drop of 4 Bar, being considered at the time of choosing the ultrafiltration equipment.

The serum is discharged directly from the lung tanks to the ultrafiltration system by means of a bank of valves and the corresponding pump or is directly fed to the balance tub of the ultrafilter.

The device in this case Is WPC 35, which will be sent to the corresponding tanks for further processing and obtaining WPC 80 according to the already existing processes.

The concentration of protein is 34% with a solids content of 9%.

The permeate will contain the lactose, which is the source of carbon that will allow further development of the yeast (Kluyveromyces marxianus), the permeate having a protein content of 3 to 5% and with a total of 5.5% solids depending on the quality of the serum (sweet whey or acid serum).

6. Fermentation:

The permeate will be sent to the fermenters considering that at the outlet the product is sterile, this medium will be enriched (inorganic salts) and acidified to pH 4.6 (phosphoric acid) in line according to the corresponding formula, nisin should be added in order to inhibit any bacterial development.

One embodiment of the present invention is a batch process, where it can be chosen by a batch process fed or a continuous fermentation for the purpose of optimizing the processes.

The fermentation process is carried out in a period of thirty hours, with 1% being inoculated. The process of the generation of the inoculum is mentioned below.

The factors to be controlled are at this critical control point, are the pH should be maintained in a range of between 4.0 and 5.5, the temperature should be maintained at 38° C., the air feed should be 1.2 volumes of air per liter of medium per minute, and it should be filtered through absolute filters of 0.21 microns and should not contain oil, that is, it has to be sanitized air, the generation of foam is controlled by means of a silicone antifoam, the agitation must be maintained on the order of 150 rpm, it is preferred that the weight of the fermenter be marine type of 8 blades and spaced from 1.5 distance diameters.

7. Collection of Biomass:

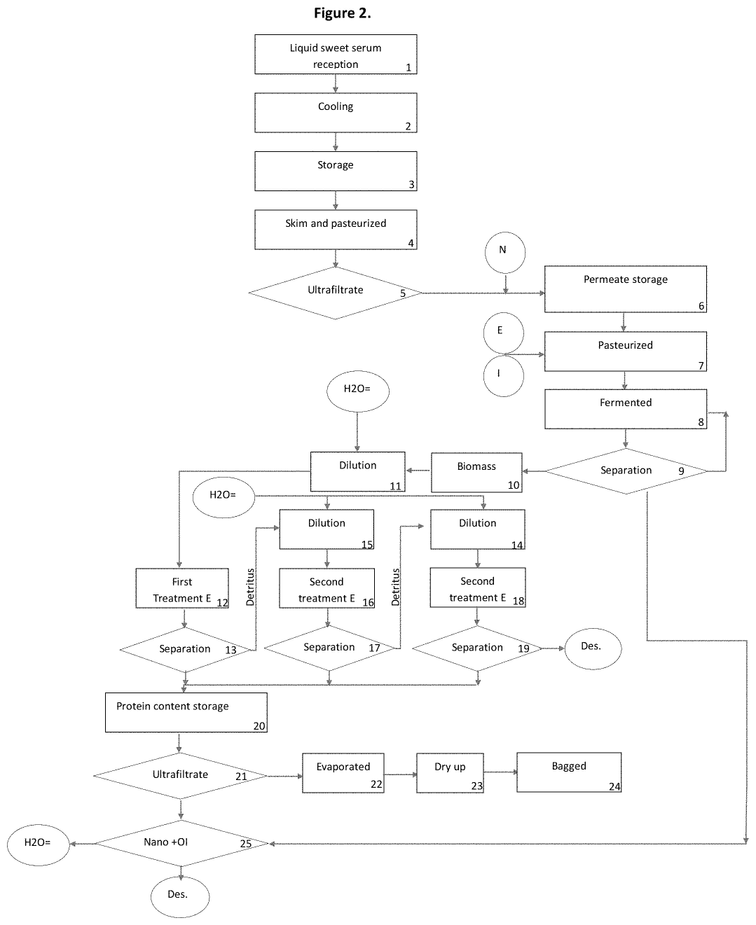

In the case of FIG. 2 or process diagram using liquid fresh serum, the process omits the generation of whey protein concentrate also known as WPC, in fact, in the description of the diagram or FIG. 1, as just mentioned at point 5 which is sent to storage tanks, which point is the only distinct point, since from this point both processes are the same.

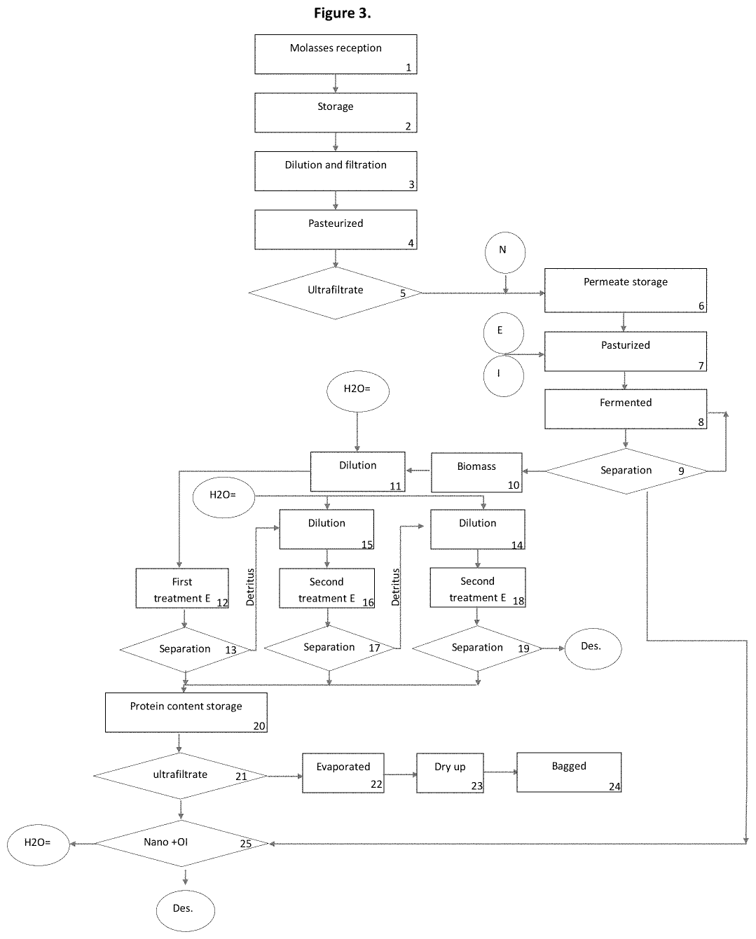

In the case of FIG. 3, the plant process begins by receiving the molasses, then diluted, clarified and passed through an activated carbon system to precisely follow the path of process B.

In fact, the formulation is the same but only sugar is changed for lactose.

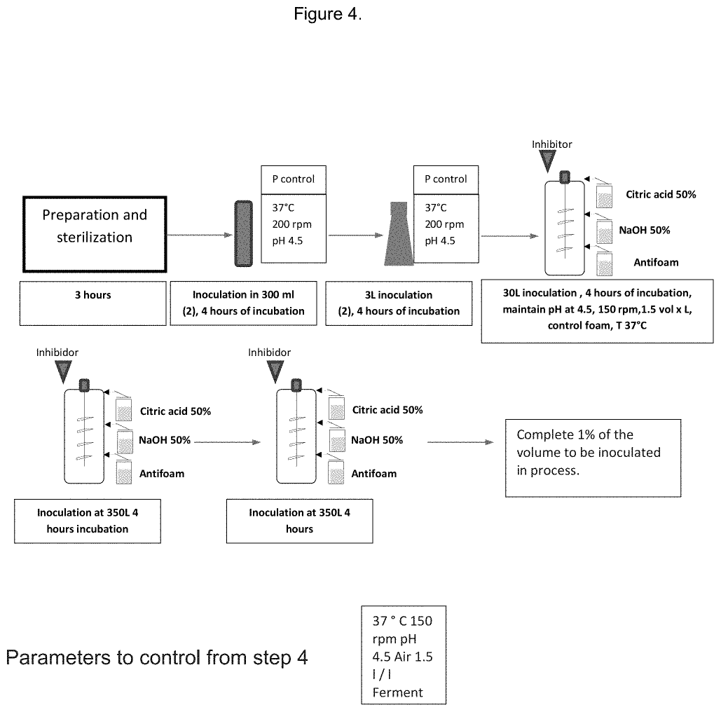

With respect to the diagram or FIG. 4, the propagation of the inoculum is illustrated, this process consists of a pure strain of Kluyveromyces marxianus, preserved in glycerol at −80° C., is thawed and poured into test tubes with 10 ml of potato dextrose medium and placed in an orbital incubator, and after two hours it is poured into a 100 ml flask and re incubated in an orbital incubator At 38° C. during four hours, then poured into a 1 liter flask and the operation is repeated to arrive at the flasks of 5 liters Ferbach, maintaining 4 hours the incubation. At the end of this operation, it must be counted with 10 liters of inoculum which can be added to the serum or molasses culture medium and will represent 1% of the total volume or to a volume of 1000 liters, which should be incubated for 8 hours and which can be used as an inoculum for 10,000 liter reactors.

This operation will be repeated batch by batch.

### Example 1

The invention relates to a test for the production of protein at IBT UNAM (Biotechnology Institute of the National Autonomous University of Mexico).

The formulation was prepared and calculated in order to establish the requirements during the development of the test, the objective was to confirm that reported by the literature in relation to the fermentation of lactose through Kluyveromyomyces marxianus in an enriched medium in order to achieve the production of protein by an enzymatic method previously analyzed and proposed. It is noted that according to theoretical research the expected yield of biomass corresponded to 50% of the lactose present. However, nitrogen sources are added to allow for a better harvest of the biomass.

As a first point, a glycerol is obtained which is at −40° C. and which contains the corresponding microorganism strain from it. It is left to the environment as the media is prepared and sterilized.

The inoculum production process was modified to be in the following manner:

Thawing of glycerol, dividing into two parts, adding to a volume of medium prepared from 100 ml, incubation at 37° C., in orbital incubator, with an incubation time of 12 hours.

The formula used for such a purpose was as follows:

| | | |
|---|---|---|
| Lactose | 20 | grams |
| ammonium Sulfate | 3 | grams |
| magnesium Sulfate | 1 | gram |
| Yeast Extract | 3 | grams |
| Potassic mono phosphate | 3 | grams |
| Urea | 2 | grams |
| Water | 1.97 | liters |

| | |
|---|---|
| Total 2 liters. | Concentration of solids 1.66% |

Although it was found slightly under the solids content, this is more related to the fact that there is a split in the production of an artificial serum in order to obviate in the process the precipitation and separation of the protein involving a more complicated process according to the circumstances of the laboratory.

One of the consequences of the sterilization and which is necessary to take into account, prior to inoculation of the culture, is that the pH was increased, in this case it passes from 4.2 to 7.2, which makes it necessary to correct the conditions of the medium in order to achieve an acidity of at least 4.5.

The aim of maintaining this pH is to prevent the development of bacteria which can modify the fermentation pathway at the time of inoculation despite the care that can be obtained.

After 12 hours the culture was checked under a microscope where the proper development of the strain was observed, it is noted that one of the practical parameters in order to verify during the development that the product is being multiplied is the appearance of a halo in the neck of the 250 ml flask.

Once this was done, the rest of the medium that was previously prepared and sterilized in the Fernbach flasks 900 ml each was used since the propagation was done in duplicate, inoculated and sent to an orbital incubator maintaining the same process conditions, that is 150 rpm stirring, 37° C. incubation and 12 hours of fermentation.

When reviewing it before the microscope, the appearance of filiform bacteria that compromise, according to laboratory observations, the viability of the test is observed. Likewise, the aroma that emerges from the product is not characteristic of yeast fermentation and if it is fermented with bacteria.

Although the recommendation was to abort the process, however, considering the growth medium in which the bacteria grown it was decided to continue with the 30 liter fermentation, adjusting the pH parameters, the 30 liter fermenter was then inoculated, which already contains the sterilized medium which was prepared according to the following formulation:

| | | |
|---|---|---|
| Lactose | 300 | grams |
| Ammonium Sulfate | 45 | grams |
| Magnesium Sulfate | 15 | grams |
| Yeast Extract | 45 | grams |
| Urea | 30 | grams |
| Water | 28 | liters |

(considering that the 2 Liters of the culture prepared would be inoculated.

To ensure the success of the process, the Fernbach was chosen with less presence of filiform organisms and inoculated to the 30-liter ferment, which should be fermented for 8 hours before final inoculation in the 30-liter fermenter. This implies that the final amount in the 30-liter fermenter was 29 liters instead of 30 liters.

Again, the medium has to be adjusted to pH 4.2, after sterilizing is increased to 6.60 of pH so that prior to inoculation the pH is adjusted to 4.03. The fermenter used was a Sartorius Biostat C Plus, fixing the operating conditions according to the following, first of all, it was established that there was no air injection conditions so that oxygen saturation was handled according to the agitator and control of the equipment. The following conditions of operation are set: agitation 200 rpm, pH 4.2±0.7, the pH was adjusted with 10% soda, as the fermentation will cause the fermentation to be acidified, at this time the saturation of oxygen was 98.2% and the temperature of incubation was set at 37° C., as well as the foaming was controlled with a the invention relates to a silicone based antifoam agent which is pre diluted to 30% to prevent clogging of the feeding systems.

After 4 hours the development of the fermentation is monitored under microscope obtaining the expected results, the presence of filiform organisms has disappeared and a correct development of yeast in the medium is observed. Thus, the fermentation process was decided to terminate.

Upon reaching 8 hours, the reading of the panel of the equipment indicates a pH of 3.69, oxygen saturation of 60% with variations at 12.1%, same as the equipment compensates, incubation temperature 37° C., a consumption of 21 ml of antifoam and base 10% NaOH 10 ml.

The odor of the fermentation medium is already correct and characteristic to a yeast fermentation, when observing the sample under a microscope it is determined that the culture is going well so that the last fermentation phase was prepared.

An optimal fermentation time of between 18 and 20 hours was determined.

Inoculate were prepared to develop new glycerols from plaque, since the cultures are clean, they were ready for the next day, and produce new glycerols.

The medium is prepared according to the following formulation:

| | | |
|---|---|---|
| Lactose | 2.700 | kilograms |
| ammonium Sulfate | 0.405 | kilograms |
| Magnesium Sulfate | 0.135 | kilogramos |
| Yeast extract | 0.405 | kilograms |
| Potassic mono phosphate | 0.405 | kilograms |
| Urea | 0.270 | kilograms |
| water | 265.680 | liters |

Again despite the medium was adjusted to 4.2 of pH, the pH after sterilization was increased to 6.60 so that prior to inoculation the pH was adjusted to 4.03, considering the volume the volume was decided to lower a little plus the pH in order to favor the development of the yeast which as reported in the literature has an optimum growth range of 3.8 to 5.0 A minimum range or lower ceiling of 3.8 was left and a high ceiling of 4.5.

The fermenter in this case is 300 liters, is fed by compressed air through absolute filters of 21 microns and has inputs for the use of sanitary steam, as well as a vapor jacket for temperature increase. It is a subject to pressure vessel, thereby regulating the operating pressure of the air of the equipment which was left to 1 bar and the product was fed via a 30 I fermenter hose through the use of pressurization with sanitary air.

In this stage there is nothing left but to wait for 20 hours of fermentation, which were fulfilled, however, at the time of transferring the product the smell of the fermented medium is extremely pleasant and has been corrected. According to the oxygen consumption curve of the fermentation, it was decided to stop at 15 hours after starting, taking into account the decrease in oxygen demand which we consider an indirect indicator of growth.

It was centrifuged in a closed centrifuge, operating speed 14,000 rpm, which processes 8 liters per minute 480 liters per hour, the product obtained as concentrated paste, dark color and pleasant smell at this stage having a characteristic taste as well as pleasant taste.

This was diluted by adding 7 liters of water to 2.9 kilograms of biomass in order to allow handling in the equipment and with a solids content corresponding to 9.28%.

This process stage was modified as a result of the replacement of the Granular Neutral Cellular enzyme by a national that acts under different conditions than those reported, adding in an amount of 1 g per kilogram of biomass. Prior to this the medium was adjusted with sodium hydroxide dissolved at 50 v/v, the product was heated to 60° C., keeping for 60 minutes at a pH of 6.8, at the end of which the temperature was lowered to 50° C., the pH was adjusted to 5.5 and the enzyme was added leaving it to react for 15 minutes, maintaining a stirring of 220 rpm.

The mixture was centrifuged to obtain a total of 8.5 liters, with a solid's concentration of 3.0%, the debris being recovered and re suspended leaving at the end a solids concentration of 9.83% to proceed to the second breaking step.

Again, the liquid is heated inside the 10-liter fermenter Sartorios, maintaining a stirring of 220 rpm, temperature of 60° C. and pH 6.8 which was adjusted with 20% dissolved hydrochloric acid. After 60 minutes, the pH was adjusted to 9.0 and the temperature at 80° C., adding only the enzyme Alkaline Protease Conc., Since the Granular Neutral Cellulase was incompatible given the pH of the medium, it should be remembered that this enzyme was substituted.

After 15 minutes, it was centrifuged again, generating a clarified with a concentration of 2.86% solids and a final volume of 7.5 liters. Again the biomass is re-suspended to have a liquid whose solids content is 9.48% and the third enzymatic treatment was carried out, it was carried out as follows: the liquid was placed in the 10 l fermenter and the operating parameters 220 rpm, pH 4.5 (again with 20% hydrochloric acid soda) to hold for 60 minutes at 60° C., then the pH was modified to 9, with 50% dissolved sodium hydroxide and raised the temperature at 80° C. to add the enzyme Cellulase Conc. AL2X. After 15 minutes it was harvested, discarding the detritus and producing 8.5 l of clarified. The solids content on this occasion was 2.7%.

In all three cases the clarified was passed through Westfalia clarifier in order to eliminate all suspended solids.

In order to take advantage of the time it is decided to start the drying of the first enzymatic extraction, initially feeding at 130° C. and with an outlet of the dryer of 60° C., in a Niro drier dryer.

Consuming the first half of the concentrate, it was observed that the protein production was very low, in fact, it corresponded to 22 g. It was decided to continue with the operation by adjusting the inlet drying temperature to 140° C. and with an output of 71° C., the performance improved, but at the time of opening the receptacle it was possible to define that the drying was inadequate since all the caking was caked protein. It was decided to stop.

Results:

The initial yield in relation to the lactose content was 1 g of wet biomass per 1.7 g of lactose 0.34 g of biomass per g of lactose. It was found that a factor that affected was the concentration of soluble oxygen, which could not be regulated in the equipment used and therefore three fermentations were required to achieve the highest productivity.

Also, it was observed that one of the important consequences of the process is the removal of solids and contaminating solutes.

The replacement of beta glucanase showed good results since the final solids content in the protein concentrate adhered to the results reported in the literature. However, given the inactivation pH, it could not be applied in the second stage, so it is advisable to recover the original enzyme.

The drying problem is correlated to the same situation apparently given the presence of lactose which prevents its drying. The two second extractions perform better with the noticeable decrease in lactose content, it is desirable that a warm water wash be provided to the biomass prior to enzyme treatments. Also, the fermentation was defined on three times.

The remaining protein was recovered in drying, leaving 1 liter of sample, the pH adjustment was requested at 7.01-7.2, which makes it more congruent to the food product.

The protein is salty, so it is recommended to reconsider the acids, changing from hydrochloric acid to citric and neutralizing with potassium hydroxide or calcium hydroxide instead of sodium hydroxide, to avoid a high sodium chloride content.

It is also important to define the lactose or solids content after the biomass harvest. If low, no additional fermentation is necessary.

The drying temperature will be adjusted upwards by stipulating 180° C. inlet and 100° C. outlet.

The possibility of obtaining the protein by lyophilizing process should also be considered. However, according to the final drying results, the product can be worked without problems in the spray drying system, so we must consider whether the lyophilizate competes with the drying.

There was an important difference between the spray product and the lyophilizate related to the density of the powder, which was greater in the lyophilizate and on the other hand the dispersibility, the freeze-drying solution of the lyophilizate was 15 times higher than the powder obtained by spray.

A test was performed where samples were obtained to determine the amino acid content of the samples obtained by the process of the present application, and compared to patterns having the results shown below for the samples individually and a comparison with patterns.

The Amino Acid Profile was determined from two samples identified as: unicellular protein, white (INN L14668) And dark unicellular protein (INN L14669), which were delivered To the National Institute of Medical and Nutrition Sciences Salvador Zubirán, for testing.

### Example 2

### Amino Acid Profile Performed to Samples of White Unicelular Protein and Food Obscure Unicelular Protein 170

Sample: unicellular, White (INN L14668) protein

### Report: 034/16/L

### Reception: 2016-02-16

### Analysis: 2016-02-19

| Amino acids | Result. | |
|---|---|---|
| profile. | g/100 g of protein. | Reference. |
| Indispensable | | |
| Triptófano ** | 2.435 | |
| Valine | 4.985 | |
| Isoleucine | 4.865 | |
| Threonine | 5.088 | |
| Phenylalanine | 3.614 | |
| Leucine | 12.175 | Internal method. Ion exchange |
| | | chromatography. MME-AA-01 |
| Lysine | 11.735 | |
| Methionine* | 1.554 | |
| Dispensable. | | * Internal method. Ion exchange |
| | | chromatography. MME-AA-02 |
| Histidina | 2.460 | |
| Aspartic acid | 10.677 | |
| Serina | 4.322 | ** Internal method. Ion exchange |
| | | chromatography. MME-AA-03¹ |
| Glutamic acid | 16.287 | |
| Proline | 3.801 | |
| Glycine | 2.581 | |
| Alanine | 4.210 | |
| Cisteic* | 2.636 | |
| Tyrosine | 3.438 | |
| Arginine. | 2.862 | |

The test results refer only to the sample analyzed.

Accredited method Accreditation No.: A-0099-007/11, effective as of 2011-11-23.

TABLE 3
COMPARATIVE CONTENT OF FOOD ANIMOACID
170 vs. DISVERSE PATTERNS.

| | | | | | | | | | MET + |
|---|---|---|---|---|---|---|---|---|---|
| Source. | LEU | LIS | SER | TREO | VAL | ILE | TRI | MET | CIST |
| ALIMENTARIA | 6.67 | 8.35 | 3.77 | 4.85 | 5.12 | 4.12 | 1.09 | 0.60 | 1.20 |
| 100 | | | | | | | | | |
| ALIMENTARIA | 12.18 | 11.74 | 4.32 | 5.08 | 4.99 | 4.87 | 2.44 | 1.554 | 4.19 |
| 170 | | | | | | | | | |
| DESCREMED | 3.41 | 2.70 | 2.10 | 1.60 | 2.40 | 2.20 | 0.49 | 0.86 | 1.37 |
| POWDER | | | | | | | | | |
| MILK | | | | | | | | | |
| EGGS | 9.00 | 6.70 | 7.70 | 5.30 | 7.20 | 5.80 | — | — | — |
| WHEAT | 6.40 | 2.70 | 4.80 | 2.90 | 4.30 | 3.80 | — | — | — |
| FAO PATTERN | 7.00 | 5.50 | 4.00 | 4.60 | 5.00 | 4.00 | 1.00 | — | 3.50 |
| (2002) | | | | | | | | | |

### Example 3

A test was performed with 270 liters.

### Materials:

| Raw Materials | amount | Units |
|---|---|---|
| Lactose | 3.000 | kg |
| Ammonium sulphate | 0.450 | kg |
| Magnesium sulphate | 0.150 | kg |
| Urea | 0.300 | kg |
| Yeast Extract | 0.450 | kg |
| Monosodium Phosphate | 0.450 | kg |
| Water | 265.200 | liter |
| Total | 270.000 | liter |

Method:
1. Culture medium was prepared.
2. S.T. was verified. The pH was adjusted to 4.5 using 10% hydrochloric acid.
3. Pasteurize at 85° C. for 60 seconds.
4. The temperature was adjusted to 35° C.
5. 10% culture was inoculated.
6. Fermentation parameters were adjusted, 100 rpm, air 1.22 VVm, pH 4.5, (the optical density is determined), and maintained at 40° C.
7. It was allowed to ferment 20 hours, using antifoam, soda and 10% hydrochloric acid to regulate pH.
8. Agitation and aeration were stopped, allowing to stand 15 minutes.
9. The biomass is centrifuged and the biomass is obtained.
10. The biomass is retained and the broth was discarded.
11. The biomass was adjusted with the weight equivalent in 10% ST water (diluting with drinking water).
12. The pH was adjusted to 6.8 with 10% V/V sodium hydroxide.
13. The temperature was raised to 60° C. during 60 minutes, constantly stirring
14. The pH was increased to 9.0 with 10% sodium hydroxide
15. The granular neutral cellulase (1 g/kg of biomass), diluted to 10% in drinking water, was added.
16. The temperature was raised to 80° C. and maintaining 15 minutes.
17. It is centrifuged, the biomass and broth are stored
18. The biomass is diluted with 1/1 drinking water, adjusting pH to 6.8 with 10% hydrochloric acid.
19. The pH was adjusted to 6.8 with 10% V/V sodium hydroxide
20. The temperature was raised to 60° C. during 60 minutes, constantly stirring
21. The pH was increased to 9.0 with 10% sodium hydroxide
22. Granular neutral cellulase and alkaline protease were added both at 1 g/per kilogram of biomass, diluted to 10% in drinking water.
23. The temperature was raised to 80° C. and held for 15 minutes.
24. It was centrifuged. biomass and broth were stored
25. The biomass was diluted with 1/1 drinking water, the pH was adjusted to 4.5 with 10% hydrochloric acid
26. The temperature was raised to 60° C. during 60 minutes, constantly stirring
27. The pH was increased to 9.0 with 10% soda
28. AL2X concentrated cellulase (1 g/per kilogram of biomass) was added, diluted to 10% in drinking water the temperature was raised to 80° C. and held for 15 minutes.
29. The temperature was raised to 80° C. and maintained for 15 minutes.
30. It was centrifuged and the broth was stored and the solids were discarded.
31. It was passed to an ultrafilter and concentrated 4 to 1, with 5 KD membranes.
32. Once having all the broth collected, the protein and solids content was determined to dry
33. It was dried at 180-200° C. and a fine powder with 4% humidity was obtained.

### INDUSTRIAL APPLICATION

A process to ferment lactose and molasses, to obtain unicellular biomass, which allows to obtain a high protein product, and uses as a means of propagation whey of cheese or molasses, for the use by Kluyveromyces marxianus for the obtaining unicellular biomass and subsequently unicellular protein, and the product directly obtained from the process are concepts that clearly meet the requirement of industrial application.

It is noted that, in relation to this date, the best method known by the applicant to implement said invention is that, which is clear from reading the present description of the invention.

Although the invention has been described in some detail by way of illustration and examples for purposes of clarity of understanding, it will be readily apparent to those of ordinary skill in the art, in light of the teachings of this invention that changes and changes can be made and modifications to it without departing from the spirit or scope of the description therein, including the appended claims.

Having sufficiently described my invention, I consider as a novelty and therefore claim as my exclusive property, the content of the following clauses:

1. A process to ferment lactose and/or molasses to obtain unicellular biomass, from sweet whey, acid, or any fermentable sugary liquid or high starch waters, using Kluyveromyces marxianus, comprising the following steps:
(1.) Serum reception;
(2.) Send to the cooling press where the temperature is lowered to approximately 4° C.;
(3.) The product is kept in the silos with constant agitation and at approximately 4° C.;
(4.) The product is discharged through the valve bank and the pump to be sent to the pasteurizer balance tank, where it will skim and take 85° C. for approximately 15 seconds;
(5.) The product is sent to the ultrafiltration membrane balance tank;
(6.) The permeate is sent to the storage tanks, and 50% w/v nutrients are added through the feed line to the storage tanks;
(7.) The product from the storage tanks is sent to a tank through a pump and from the valve bank to the plate exchanger balance tank to bring the product to 85° C. for 1 min and lower the temperature to approximately 40° C.;
(8.) The product is sent to the fermenters, where the equipment is prepared to control the volume of air to be injected that must be maintained in a ratio of approximately 1.5 liters of air per liter of sugar dilution, the pH range 5.6 to 4.6, and wherein the stirring will be maintained at approximately 200 rpm, the temperature will be in the range of 39 to 41° C., and the generation of foam is controlled through the dosing of silicon dioxide, the medium is acidified with a phosphoric acid solution and is basified with a 50% sodium hydroxide solution;
(9.) The product is sent by pumping to the decanter or centrifuge by means of nozzles so that the product is separated into biomass and clarified;
(10.) The clarified is sent to the storage tank from which the equivalent of 5% of the volume of production is pumped to the balance tub, the rest is stored for its last nanofiltration and reverse osmosis treatment in order to recover water;
(11.) The biomass resulting from the clarification is sent by pumping to a lung tank to be diluted through a solution of water buffered with sodium hexametaphosphate (1 g/per liter) in order to eliminate carbohydrates;
(12.) The solution is sent by pumping to be separated by decantation or by centrifuge of nozzles, from which the biomass is sent by pumping to the first enzymatic treatment and the clarified to the nanofiltration and reverse osmosis treatment;
(13.) In the enzyme treatment tank, the biomass is diluted to 10% of total solids, passing through a plate exchanger and raising the temperature to approximately 68° C.;
(14.) Once the dilution is achieved, the pH is raised to 6.8 to keep it that way for one hour and then it is passed through a plate exchanger with a pump and the temperature is raised to approximately 80° C., the pH is adjusted to 8.0 adding sodium hydroxide, 1.5 g of granular neutral cellulase is added for every Kg of dry base biomass dissolved in 10% water, keeping it for 15 minutes;
(15.) The solution is sent by pumping to separate by decantation or by centrifuge of nozzles, and the biomass is sent by pumping to the second enzymatic treatment and the clarified to the proteinized broth tank for storage, with a content of 3% of total solids;
(16.) In the second enzyme treatment tank, the biomass is diluted to 10% of total solids, passing through a plate exchanger to raise the temperature to approximately 60° C.;
(17.) Once the dilution is achieved, the pH is raised to 6.8 to maintain for one hour by passing a plate exchanger by pumping to raise the temperature to 80° C., adjusting the pH to 8.0 with the addition of Sodium hydroxide, 1.5 g of granular neutral cellulase and conc. alkaline protease are added, for each Kg of dry base biomass dissolved in 10% water, thus maintaining for 15 minutes;
(18.) The solution is sent by pumping to be separated by decantation or by centrifuge of nozzles, and the biomass is sent by pumping to the second enzymatic treatment and the clarified one to the proteinized broth tank for storage is containing 2.8% of total solids;
(19.) The biomass is diluted to 10% of total solids in the three-enzyme treatment tank, passing through a plate exchanger to raise the temperature to 60° C.;
(20.) Once the dilution is achieved, the pH is decreased to 4.5 with phosphoric acid for one hour, through a plate exchanger by pumping to raise the temperature to 80° C., adjusting the pH to 8.0 with the addition of sodium hydroxide, 1.5 g of conc. cellulase is added. AL2X, for every Kg of dry base biomass, keeping it for 15 minutes;
(21.) The solution is sent by pumping to be separated by decantation or by centrifuge of nozzles, and the biomass is sent by pumping to the collection vessel and the clarification to the proteinazed broth tank for storage;
(22.) In the storage tank the content of solids and protein is verified, the pH is adjusted between 6.8 to 7.0, with sodium hydroxide or phosphoric acid as appropriate and maintained with stirring; and is then sent to the membrane filtration system to remove salts and increase protein content;
It is sent by pumping to the ultrafiltration system;
(23.) The detainee is sent to a lung tank where the content of protein, carbohydrates, salt and pH is verified, immediately going to nanofiltration;
(24.) The next step is electrodialysis while eliminating nucleic acids, and at this stage pasteurization is performed, once this is done it is sent to the evaporation process;
(25.) The concentrated product is spray dried at an inlet temperature of 160 and up to 180° C. and at an outlet temperature of between 60 to 80° C.;
(26.) It is finally bagged and stored; and
(27.) Of the ultrafiltration and nanofiltration systems, the retention is sent by pumping to a lung tank where it is kept to evaporate, at this stage it is heated by plate exchange at 60° C., the permeate is sent to the tank lung to be able to accumulate with the rest of the clarification of the biomass and the water of washing of biomass in order to be sent later to ultrafiltration and reverse osmosis.

2. The process to ferment lactose and/or molasses to obtain unicellular biomass, from sweet whey, acid, or any fermentable sugary liquid or high starch waters, using Kluyveromyces marxianus, according to claim 1, wherein in step 6) the addition of nutrients is done according to the following relationship:

| Name | amount. |
|---|---|
| Lactose | 1.00 |
| Ammonium sulphate | 0.15 |
| Magnesium sulphate | 0.05 |
| Yeast extract | 0.15 |
| Mono Potassium Phosphate | 0.15 |
| Urea | 0.1 |
| Water | 98.4 |
| Total. | 100.00 |

3. The process for fermenting lactose and/or molasses to obtain unicellular biomass, from sweet whey, acid, or any fermentable sugary liquid or high starch waters, using Kluyveromyces marxianus, according to claim 1, wherein when the raw material is molasses, the process begins upon receiving the molasses, subsequently diluted, clarified and passed through an activated carbon system, and then the rest of the process continues without change.

4. The process for fermenting lactose and/or molasses to obtain unicellular biomass, from sweet whey, acid, or any fermentable sugary liquid or high starch waters, using Kluyveromyces marxianus, according to claim 1, wherein the process can begin with the propagation of the inoculum, starting from a pure strain of Kluyveromyces marxianus, preserved in glycerol at −80° C., it is thawed and poured into test tubes with 10 ml of potato dextrose medium and placed in an orbital incubator, after two hours it is poured into 100 ml flasks and re-incubated in an orbital incubator at 38° C. for four hours, then it is poured into a 1 liter flask and the operation is repeated, until reaching 5 liter flasks, maintaining incubation for 4 hours.

5. The process for fermenting lactose and/or molasses to obtain unicellular biomass, from sweet whey, acid, or any fermentable sugary liquid or high starch waters, using Kluyveromyces marxianus, according to claim 1, wherein in step 8) if the process is batching the fermentation will be carried out for 20 hours.

6. The process to ferment lactose and/or molasses to obtain unicellular biomass, from sweet whey, acid, or any fermentable sugary liquid or high starch waters, using Kluyveromyces marxianus, according to claim 1, wherein in step 8) if the fermentation process is continuous the replacement rate will be 20%.

7. A protein product obtainable directly from the process according to claim 1.

8. A use of the protein product obtainable directly from the process according to claim 1, wherein said protein product is used as a complement to dairy, flour and fruit drinks.

9. A protein product obtainable directly from the process according to claim 1, wherein said protein product has the following amino acid content:

| Amino acids | Result. |
|---|---|
| profile. | g/100 g of protein. |
| Indispensable | |
| Tryptophan** | 2.435 |
| Valine | 4.985 |
| Isoleucine | 4.865 |
| Threonine | 5.088 |
| Phenylalanine | 3.614 |
| Leucine | 12.175 |
| Lysine | 11.735 |
| Methionine* | 1.554 |
| Dispensable | |
| Histidine | 2.460 |
| Aspartic acid | 10.677 |
| Serine | 4.322 |
| Glutamic acid | 16.287 |
| Proline | 3.801 |
| Glycine | 2.581 |
| Alanine | 4.210 |
| Cisteic * | 2.636 |
| Tyrosine | 3.438 |
| Arginine | 2.862 |
